(19) United States
(12) Patent Application Publication
(10) Pub. No.: US 2010/0087759 A1
(43) Pub. Date: Apr. 8, 2010

(54) APPARATUS AND METHOD FOR AN ULTRASONIC MEDICAL DEVICE WITH VARIABLE FREQUENCY DRIVE

(76) Inventors: Bradley A. Hare, Chelmsford, MA (US); Kyle K. Jarger, Stow, MA (US); Charles J. Vadala, JR., Roslindale, MA (US); Anthony W. O'Leary, Walpole, MA (US); Thomas A. Murphy, Malden, MA (US); Roy M. Robertson, Saugus, MA (US)

Correspondence Address: Omnisonics Medical Technologies, Inc.;c/o John Aquino (Bankruptcy Trustee),, Anderson Aquino LLP, 240 Lewis Wharf, Boston, MA, 02110, US

(21) Appl. No.: 12/555,495

(22) Filed: Sep. 8, 2009

Related U.S. Application Data
(63) Continuation of application No. 10/999,539, filed on Nov. 30, 2004, now abandoned.

(51) Int. Cl. A61N 7/00

(52) U.S. Cl. 601/2

(57) ABSTRACT

An apparatus and method for an ultrasonic medical device with a variable frequency drive for ablating a biological material comprises an ultrasonic probe having a proximal end, a distal end and a longitudinal axis therebetween; a transducer that drives the ultrasonic probe over a variable frequency range, creating a transverse ultrasonic vibration along at least a portion of the longitudinal axis of the ultrasonic probe; a coupling engaging the proximal end of the ultrasonic probe to a distal end of the transducer; and an ultrasonic energy source engaged to the transducer that produces an ultrasonic energy, wherein driving the ultrasonic probe over the variable frequency range allows for the ultrasonic energy to propagate around a bend of the ultrasonic probe to ablate the biological material in communication with the ultrasonic probe.

### CROSS-REFERENCE TO RELATED APPLICATIONS

This application is a continuation application of and claims priority to U.S. application Ser. No. 10/999,539, filed on Nov. 30, 2004, which is incorporated by reference herein.

### FIELD OF THE INVENTION

The present invention relates to medical devices, and more particularly to an apparatus and a method for an ultrasonic medical device with a variable frequency drive to ablate a biological material.

### BACKGROUND OF THE INVENTION

The body's transport system is a complicated network of vasculatures that includes, but is not limited to, arteries, veins, vessels, capillaries, intestines, ducts and other body lumen. Blood travels around the body in over seventy-five thousand miles of the vasculatures, which when stretched end to end is a length approximately equivalent to three times around the world. The vasculatures of the body transport oxygen from the lungs, remove carbon dioxide from the cells and carry nutrients, hormones and water to all parts of the body.

The vasculatures throughout the body bend to perform the various functions which they serve. For example, the circulation in the body is a closed loop of vasculatures that run in an approximately continuous figure eight centered around the heart. As an example, the heart is a double circulation system from which pulmonary arteries and pulmonary veins move in and out of by bending around various organs within the body. The pulmonary arteries carry blood away from the heart to the lungs while the pulmonary veins bring blood from the lungs to the heart.

In many medical procedures, a medical device is inserted into the vasculature and navigated to a treatment site. The bends within the vasculature make it more difficult to maneuver the medical device to the treatment site. In addition, the bends within the vasculatures can affect the functionality of the working portion of the medical device, thereby requiring special design to the medical device.

U.S. Pat. No. 5,895,997 to Puskas et al. discloses a frequency modulated ultrasonic generator for driving an ultrasonic transducer for use in ultrasonic cleaning. The Puskas et al. generator is capable of maintaining substantially constant real output to a load while the output frequency of the generator is a square wave frequency modulated about a wide bandwidth. Since the Puskas et al. device is limited to operating between two different frequencies, the ultrasonic effects of the Puskas et al. device are limited. The Puskas et al. device operates in a limited range and does not comprise any mechanisms to find particular resonances and avoid other resonances.

U.S. Pat. No. 5,452,611 to Jones et al. discloses an ultrasonic level instrument with dual frequency operation. The Jones et al. device comprises an excitation circuit that simultaneously induces vibrations at a first and a second frequency in a transmitting piezoelectric crystal, with the vibrations detected by a receiving crystal. The Jones et al. device utilizes a very resonant piezoelectric crystal that is operated with a pulse and resonates at several frequencies simultaneously.

The prior art does not provide a solution for providing uniform power output to an ultrasonic medical device to compensate for power loss incurred when bending the ultrasonic medical device through the tortuous paths of the vasculature. Prior art instruments do not provide a solution for driving an ultrasonic medical device over a variable frequency range to allow ultrasonic energy to propagate around a bend of the ultrasonic medical device. Therefore, there remains a need in the art for an apparatus and a method for ablating a biological material when the ultrasonic medical device is in a bent configuration that is effective, safe, reliable and provides a uniform power output to ablate the biological material.

### SUMMARY OF THE INVENTION

The present invention provides an apparatus and a method for using an ultrasonic medical device over a variable frequency range to allow ultrasonic energy to propagate around a bend of the ultrasonic medical device to ablate a biological material. An ultrasonic probe of the ultrasonic medical device is inserted in an insertion point of a vasculature and navigated around one or more bends of the vasculature and placed in communication with a biological material. A transducer of the ultrasonic medical device can drive the ultrasonic probe over a broad frequency range to excite the transverse resonances of the ultrasonic probe and maximize the biological material destroying effects of the ultrasonic probe. An effective zone of ablation of the biological material is increased by changing the operating frequency of the ultrasonic medical device of the present invention.

An apparatus for an ultrasonic medical device with a variable frequency drive for ablating a biological material comprises an ultrasonic probe having a proximal end, a distal end and a longitudinal axis therebetween; a transducer that drives the ultrasonic probe over a variable frequency range, creating a transverse ultrasonic vibration along at least a portion of the longitudinal axis of the ultrasonic probe; a coupling engaging the proximal end of the ultrasonic probe to a distal end of the transducer; and an ultrasonic energy source engaged to the transducer that produces an ultrasonic energy, wherein driving the ultrasonic probe over the variable frequency range allows for the ultrasonic energy to propagate around a bend of the ultrasonic probe to ablate the biological material in communication with the ultrasonic probe.

An ultrasonic medical device for resolving a biological material comprises an ultrasonic probe having a proximal end, a distal end terminating in a probe tip and a longitudinal axis between the proximal end and the distal end; a transducer that converts electrical energy into mechanical energy, creating a transverse ultrasonic vibration along the longitudinal axis of the ultrasonic probe; a coupling engaging the proximal end of the ultrasonic probe to a distal end of the transducer, wherein the ultrasonic probe is driven over a variable frequency range with an approximately uniform power output to ablate the biological material.

A method of propagating an ultrasonic energy along a bend of an ultrasonic medical device to ablate a biological material comprises providing the ultrasonic medical device comprising an ultrasonic probe having a proximal end, a distal end and a longitudinal axis therebetween; inserting the ultrasonic probe in a vasculature of a body; flexing the ultrasonic probe along a bend of the vasculature; moving the ultrasonic probe adjacent to the biological material; activating an ultrasonic energy source engaged to the ultrasonic probe to generate a transverse ultrasonic vibration along at least a portion of the longitudinal axis of the ultrasonic probe; and driving the ultrasonic probe over a variable frequency range to allow the ultrasonic energy to propagate along a bend of the ultrasonic probe to ablate the biological material.

A method of ablating a biological material adjacent to a bend in a vasculature of a body comprises providing an ultrasonic medical device comprising an ultrasonic probe having a proximal end, a distal end terminating in a probe tip and a longitudinal axis between the proximal end and the distal end; inserting the ultrasonic probe in an insertion point of the vasculature; moving the ultrasonic probe along the bend in the vasculature; placing the ultrasonic probe in communication with the biological material; activating an ultrasonic energy source engaged to the ultrasonic probe to produce an electric signal that drives a transducer of the ultrasonic medical device to produce a transverse ultrasonic vibration of the ultrasonic probe; driving the ultrasonic probe over a variable frequency range to maintain a biological material destroying effect along a bend of the ultrasonic probe.

The present invention provides an apparatus and a method for an ultrasonic medical device with a variable frequency drive for ablating a biological material. The present invention provides an ultrasonic medical device with a variable frequency drive that is simple, user-friendly, time efficient, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic medical device of the present invention being flexed around a bend in a vasculature of a body.

FIG. 2 is a side plan view of an ultrasonic probe of the present invention having a transition from a proximal end of the ultrasonic probe to a distal end of the ultrasonic probe.

FIG. 3 is a side plan view of an ultrasonic probe of the present invention having an approximately uniform diameter from a proximal end of the ultrasonic probe to a distal end of the ultrasonic probe.

FIG. 4 is a side plan view of an ultrasonic probe of the present invention showing a plurality of transverse nodes and a plurality of transverse anti-nodes along a portion of a longitudinal axis of the ultrasonic probe.

FIG. 5 is a view of an ultrasonic probe of the present invention showing a plurality of transverse nodes and a plurality of transverse anti-nodes while in communication with a biological material in a vasculature of a body.

FIG. 6 is a block diagram of a preferred embodiment of a system of an ultrasonic medical device of the present invention using phase analysis feedback.

FIG. 7 is a block diagram of an alternative embodiment of a system of an ultrasonic medical device of the present invention using spectrum analysis feedback.

FIG. 8A and FIG. 8B illustrate the effect of bending the ultrasonic probe at various locations versus energizing the ultrasonic probe at two different frequencies. FIG. 8A is a diagram showing the effect of bending the ultrasonic probe at various locations while energizing the probe at a frequency of 21 kHz. FIG. 8B is a diagram showing the effect of bending the ultrasonic probe at various locations while energizing the probe at a frequency of 23 kHz.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an ultrasonic medical device over a variable frequency range to allow ultrasonic energy to propagate around a bend of the ultrasonic medical device to ablate a biological material. An ultrasonic probe of the ultrasonic medical device is inserted in an insertion point of a vasculature and navigated around one or more bends of the vasculature and placed in communication with a biological material. A transducer of the ultrasonic medical device can drive the ultrasonic probe over a broad frequency range to excite the transverse resonances of the ultrasonic probe and maximize the biological material destroying effects of the ultrasonic probe. An effective zone of ablation of the biological material is increased by changing the operating frequency of the ultrasonic medical device of the present invention.

The following terms and definitions are used herein:

“Ablate” as used herein refers to removing, clearing, destroying or taking away a biological material. “Ablation” as used herein refers to a removal, clearance, destruction, or taking away of the biological material.

“Anti-node” as used herein refers to a region of a maximum energy emitted by an ultrasonic probe at or adjacent to a specific location along a longitudinal axis of the ultrasonic probe.

“Node” as used herein refers to a region of a minimum energy emitted by an ultrasonic probe at or adjacent to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active section” of the probe).

“Biological Material” as used herein refers to a collection of a matter including, but not limited to, a group of similar cells, intravascular blood clots, occlusions, plaque, fibrin, calcified plaque, calcium deposits, occlusional deposits, atherosclerotic plaque, fatty deposits, adipose tissues, atherosclerotic cholesterol buildup, thrombus, fibrous material buildup, arterial stenoses, minerals, high water content tissues, platelets, cellular debris, wastes and other occlusive materials.

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Vasculature” as used herein refers to the entire circulatory system for the blood supply including the venous system, the arterial system and the associated vessels, arteries, veins, capillaries, blood, and the heart. The arterial system is the means by which blood with oxygen and nutrients is transported to tissues. The venous system is the means by which blood with carbon dioxide and metabolic by-products is transported for excretion.

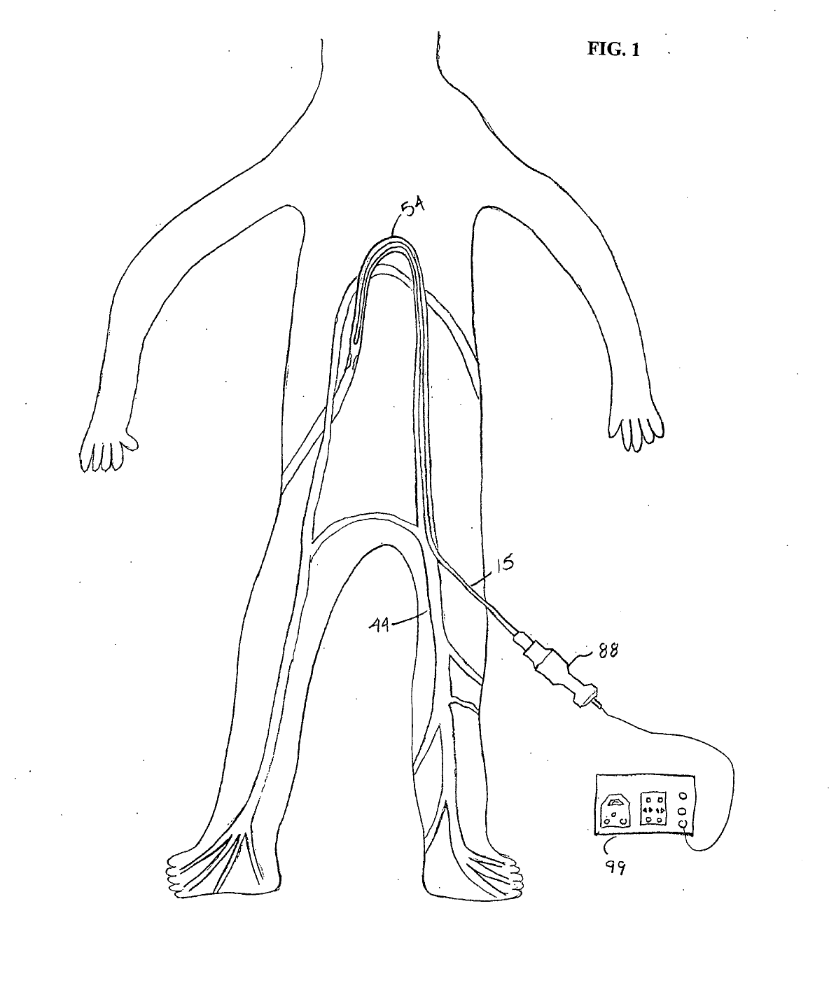

An ultrasonic probe of an ultrasonic medical device 11 with a variable frequency drive is illustrated generally at 15 in FIG. 1 being flexed around a bend 54 in a vasculature 44. The ultrasonic medical device 11 includes an ultrasonic probe 15 which is coupled to an ultrasonic energy source or generator 99 for the production of an ultrasonic energy. A handle 88, comprising a proximal end 87 and a distal end 86, surrounds a transducer within the handle 88.

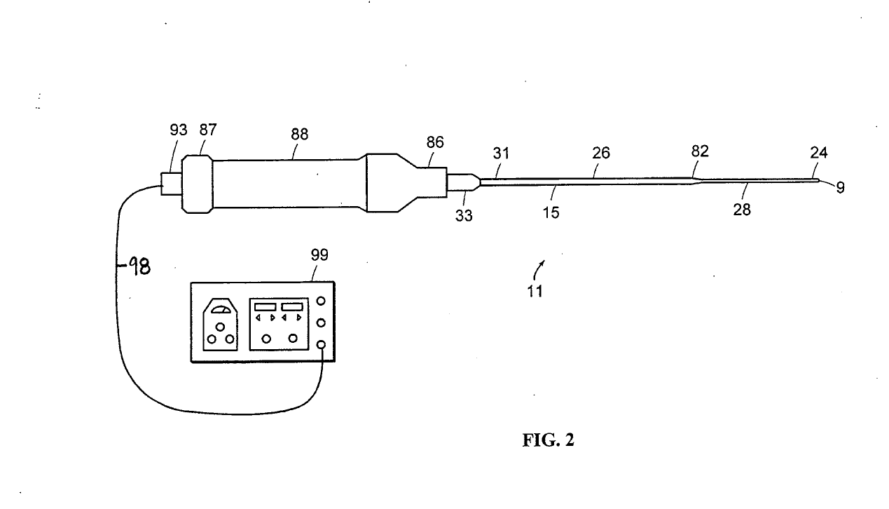

FIG. 2 shows a preferred embodiment of the ultrasonic probe 15 of the present invention where a diameter of the ultrasonic probe decreases from a first defined interval 26 to a second defined interval 28 along the longitudinal axis of the ultrasonic probe 15 over a transition 82. The ultrasonic probe 15 includes a proximal end 31, a distal end 24 that ends in a probe tip 9 and a longitudinal axis between the proximal end 31 and the distal end 24. A coupling 33 that engages the proximal end 31 of the ultrasonic probe 15 to the transducer within the handle 88 is illustrated generally in FIG. 2. In a preferred embodiment of the present invention, the coupling is a quick attachment-detachment system. An ultrasonic medical device with a rapid attachment and detachment means is described in the Assignee's U.S. Pat. No. 6,695,782 and Assignee's co-pending patent applications U.S. Ser. No. 10/268,487 and U.S. Ser. No. 10/268,843, which further describe the quick attachment-detachment system and the entirety of these patents and patent applications are hereby incorporated herein by reference.

The transducer, having a proximal end engaging the ultrasonic energy source 99 and a distal end coupled to a proximal end 31 of the ultrasonic probe 15, transmits the ultrasonic energy to the ultrasonic probe 15. The transducer is also commonly known as a driver. A connector 93 and a connecting wire 98 engage the ultrasonic energy source 99 to the transducer.

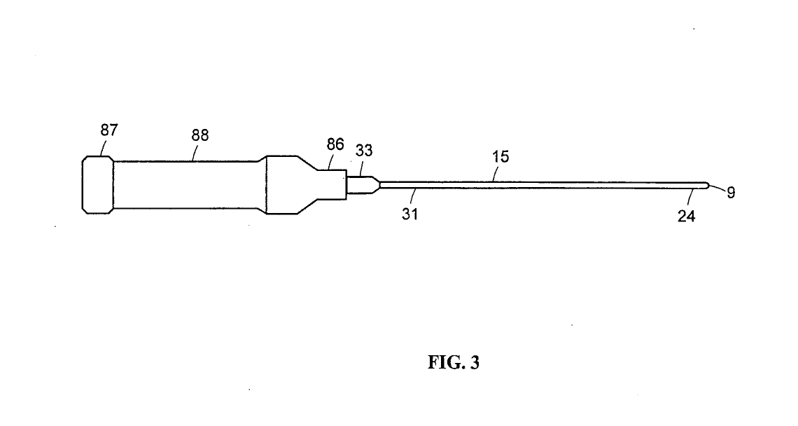

FIG. 3 shows an alternative embodiment of the ultrasonic probe 15 of the present invention. In the embodiment of the present invention shown in FIG. 3, the diameter of the ultrasonic probe 15 is approximately uniform from the proximal end 31 of the ultrasonic probe 15 to the distal end 24 of the ultrasonic probe 15.

In a preferred embodiment of the present invention, the ultrasonic probe 15 is a wire. In an embodiment of the present invention, the ultrasonic probe 15 is elongated. In an embodiment of the present invention, the diameter of the ultrasonic probe 15 changes at greater than two defined intervals. In an embodiment of the present invention, the transitions 82 of the ultrasonic probe 15 are tapered to gradually change the diameter from the proximal end 31 to the distal end 24 along the longitudinal axis of the ultrasonic probe 15. In another embodiment of the present invention, the transitions 82 of the ultrasonic probe 15 are stepwise to change the diameter from the proximal end 31 to the distal end 24 along the longitudinal axis of the ultrasonic probe 15. Those skilled in the art will recognize there can be any number of defined intervals and transitions, and the transitions can be of any shape known in the art and be within the spirit and scope of the present invention.

In an embodiment of the present invention, the gradual change of the diameter from the proximal end 31 to the distal end 24 occurs over the at least one transition 82, with each transition 82 having an approximately equal length. In another embodiment of the present invention, the gradual change of the diameter from the proximal end 31 to the distal end 24 occurs over a plurality of transitions 82 with each transition 82 having a varying length. The transition 82 refers to a section where the diameter varies from a first diameter to a second diameter.

In a preferred embodiment of the present invention, the ultrasonic probe 15 has a small diameter. In a preferred embodiment of the present invention, the cross section of the ultrasonic probe 15 is approximately circular. In another embodiment, the cross section of at least a portion of the ultrasonic probe 15 is non-circular. The ultrasonic probe 15 comprising a wire having a non-circular cross section at the distal end can navigate through the vasculature. The ultrasonic probe 15 comprising a flat wire is steerable in the vasculature. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

The probe tip 9 can be any shape including, but not limited to, rounded, bent, a ball or larger shapes. In a preferred embodiment of the present invention, the probe tip 9 is smooth to prevent damage to the vasculatures of the body. In one embodiment of the present invention, the ultrasonic energy source 99 is a physical part of the ultrasonic medical device 11. In another embodiment of the present invention, the ultrasonic energy source 99 is not an integral part of the ultrasonic medical device 11. The ultrasonic probe 15 is used to ablate biological material and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The ultrasonic probe 15 is designed, constructed and comprised of a material to operate in a transverse mode and not dampen the transverse ultrasonic vibration, and thereby supports a transverse vibration when flexed. In a preferred embodiment of the present invention, the ultrasonic probe 15 comprises titanium or a titanium alloy. Titanium is a strong, flexible, low density, low radiopacity and easily fabricated metal that is used as a structural material. Titanium and its alloys have excellent corrosion resistance in many environments and have good elevated temperature properties. In a preferred embodiment of the present invention, the ultrasonic probe 15 comprises titanium alloy Ti-6Al-4V. The elements comprising Ti-6Al-4V and the representative elemental weight percentages of Ti-6Al-4V are titanium (about 90%), aluminum (about 6%), vanadium (about 4%), iron (maximum about 0.25%) and oxygen (maximum about 0.2%). In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel.

In another embodiment of the present invention, the ultrasonic probe 15 comprises a super-elastic alloy. Even when bent or stretched, the super-elastic alloy returns to its original shape when the stress is removed. The ultrasonic probe 15 may contain super-elastic alloys known in the art including, but not limited to, nickel-titanium super-elastic alloys and Nitinol. Nitinol is a family of intermetallic materials, which contain a nearly equal mixture of nickel and titanium. Other elements can be added to adjust or tune the material properties. Nitinol is less stiff than titanium and is maneuverable in the vasculature. Nitonol has shape memory and super-elastic characteristics. The shape memory effect describes the process of restoring the original shape of a plastically deformed sample by heating it. This is a result of a crystalline phase change known as thermoelastic martensitic transformation. Below the transformation temperature, Nitinol is martensitic. Nitinol's excellent corrosion resistance, biocompatibility, and unique mechanical properties make it well suited for medical devices. Those skilled in the art will recognize that the ultrasonic probe can be comprised of many other materials known in the art and be within the spirit and scope of the present invention.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. In an embodiment of the present invention, the ultrasonic probe 15 is between about 30 centimeters and about 300 centimeters in length. Those skilled in the art will recognize an ultrasonic probe can have a length shorter than about 30 centimeters, a length longer than about 300 centimeters and a length between about 30 centimeters and about 300 centimeters and be within the spirit and scope of the present invention.

The handle 88 surrounds the transducer located between the proximal end 31 of the ultrasonic probe 15 and the connector 93. The transducer may include, but is not limited to, a horn, an electrode, an insulator, a backnut, a washer, a piezo microphone, and a piezo drive The transducer converts electrical energy provided by the ultrasonic energy source 99 to mechanical energy. The transducer is capable of engaging the ultrasonic probe 15 at the proximal end 31 with sufficient restraint to form an acoustical mass that can propagate the ultrasonic energy provided by the ultrasonic energy source 99. The ultrasonic energy source 99 provides an electrical signal to the transducer that is located within the handle 88.

A medical professional gains access to a vasculature 44 through an insertion point in the vasculature 44. A device including, but not limited to, a vascular introducer can be used to create an insertion point in the vasculature 44 to gain access to the vasculature 44. A vascular introducer for use with an ultrasonic probe is described in Assignee's co-pending patent application U.S. Ser. No. 10/080,787, and the entirety of this application is hereby incorporated herein by reference.

With access to the vasculature 44 through the insertion point in the vasculature 44, the ultrasonic probe 15 is moved adjacent to a biological material 16 in the vasculature 44. As the ultrasonic probe 15 is moved adjacent to the biological material 16, the ultrasonic probe 15 is bent through the tortuous paths of the vasculature 44. The ultrasonic probe 15 has a stiffness that gives the ultrasonic probe 15 a flexibility allowing the ultrasonic probe 15 to be deflected, flexed and bent through the tortuous paths of the vasculatures 44 of the body. The ultrasonic probe 15 can be bent, flexed and deflected to reach the biological material 16 in the vasculatures 44 of the body that would otherwise be difficult to reach. The ultrasonic probe 15 is placed in communication with the biological material 16 by moving, sweeping, bending, twisting or rotating the ultrasonic probe 15 along the biological material 16. Those skilled in the art will recognize that the many ways to move the ultrasonic probe in communication with the biological material known in the art are within the spirit and scope of the present invention.

Depending upon the ultrasonic energy source 99 and the driver, bending the ultrasonic probe 15 affects the functionality and performance of the ultrasonic probe 15. Depending upon the particular bend location and operating frequency, ultrasonic energy may not be able to propagate around the bend to allow for ablation of the biological material 16 along an active section of the ultrasonic probe 15. Instead, the operating frequency needs to be varied in order to allow the ultrasonic energy to propagate around the bend to allow for ablation of the biological material 16.

For example, prior art mechanisms utilizing a resonant driver and operating in a longitudinal mode of vibration are limited in ablation of a biological material in the body when bending the ultrasonic probe through the tortuous paths within the vasculature. Prior art mechanisms utilizing a resonant driver and operating in a longitudinal mode of vibration cannot deliver sufficient ultrasonic energy to a target area of biological material. Bending the ultrasonic probe produces a reflection from the point of maximum curvature that interferes with the driver if the driver is a resonant device. Bending the ultrasonic probe can result in the excitation of either longitudinal modes of vibration or transverse modes of vibration. If the ultrasonic probe is bent such that the reflection comes back with the right phase relationship, the reflection can either interfere with the longitudinal resonance of the driver or constructively add to the longitudinal resonance of the driver, producing an ultrasonic medical device operating in a longitudinal mode. When moving the ultrasonic probe 15 around a bend in the vasculature 44 of the body, the ultrasonic probe 15 is bent at an arbitrary location. By bending the ultrasonic probe 15 at the arbitrary location, there will be a frequency whereby a perfect standing wave pattern is created on the ultrasonic probe 15. A resonant condition is characterized by the creation of a standing wave pattern on the ultrasonic probe 15.

Prior art mechanisms are resonant systems comprising piezoelectric drivers where operation occurs at resonant frequencies of the piezoelectric drivers. With a piezoelectric driver, operation does not occur at other frequencies since sufficient physical power can not be produced at other frequencies. Prior art mechanisms have also utilized harmonics of the resonant frequency (e.g., second harmonic, third harmonic). However, operation is still at a resonant frequency, thereby only allowing for energy to be produced at or near to the resonant frequency of the piezoelectric driver.

The ultrasonic medical device 11 of the present invention comprises a variable frequency drive and operates in a transverse mode of vibration. The ultrasonic medical device 11 of the present invention comprises a transducer with the ability to drive the ultrasonic probe 15 over a wide range of frequencies, thereby producing power over a wide range of frequencies. As discussed above, prior art mechanisms utilize piezoelectric drivers that operate at resonant frequencies to drive the ultrasonic medical device. The ultrasonic medical device 11 of the present invention comprises a broadband transducer operating at various frequencies away from resonant frequencies in the ultrasonic probe 15. The ultrasonic medical device 11 of the present invention excites the transverse resonances of the ultrasonic probe 15 while avoiding the longitudinal resonances of the ultrasonic probe 15.

The ultrasonic medical device 11 of the present invention allows for variable frequency drive operation at a range of frequencies so the reflection can be controlled to not be in phase or out of phase with the driver. Thus, there is no interference with the driver. The ultrasonic medical device of the present invention allows for the frequency to be changed to avoid longitudinal resonance of the ultrasonic probe 15 and only excite transverse resonance of the ultrasonic probe 15. The ultrasonic medical device 11 of the present invention allows for the operating frequency to be varied to allow for the propagation of power around the bend to maximize the biological material ablation effects of the ultrasonic probe 15. The ultrasonic medical device 11 of the present invention allows for the operating frequency to be changed to provide delivery of adequate ultrasonic energy to ablate the biological material.

Operation of the variable frequency drive of the ultrasonic medical device 11 of the present invention is done to avoid a sparse population of longitudinal modes of vibration and preferentially excite a large population of transverse modes of vibration to maximize the biological material ablation effect. By changing the frequency, the pattern on the ultrasonic probe 15 is changed, creating the opportunity to excite the transverse mode of vibration since there are many transverse modes of vibration.

The ultrasonic medical device 11 of the present invention comprises a broadband transducer that avoids resonant frequencies in the frequency range of interest. As opposed to prior art transducers, the broadband transducer of the present invention does not have a resonance which is locked and driven on the resonant frequency. A transducer having resonances gives an uneven power output over a wide frequency range. The broadband transducer of the present invention allows for uniform power output over the frequency range the ultrasonic medical device 11 is operating through. In a preferred embodiment of the present invention, the transducer is a magnetostrictive mechanism. A magnetostrictive mechanism allows for more displacement for the same given amount of input power, allowing for a nonresonant transducer. In another embodiment of the present invention, the transducer is a voicecoil mechanism similar to what is used in a conventional audio speaker. In another embodiment of the present invention, the transducer is a pneumatic mechanism. Those skilled in the art will recognize the transducer can be many mechanisms known in the art that allow for variable frequency drive operation while avoiding any resonances in a frequency range of interest and be within the spirit and scope of the present invention.

Mechanical design of the driver avoids sharp resonances in the driver. In one embodiment of the present invention, mechanical parameters (e.g., the relative dimensions of length and diameter and pre-load stress) are chosen so that resonance at the frequency of interest is relatively flat and wide. In another embodiment, the mechanical driver is small enough or stiff enough that the acoustic resonances are higher than the drive frequency.

The ultrasonic energy source 99 of the ultrasonic medical device 11 of the present invention is a broadband ultrasonic energy source. The ultrasonic energy source of the ultrasonic medical device of the present invention is the source of electrical stimulus to the driver and itself is not resonant. The ultrasonic energy source of the ultrasonic medical device of the present invention is capable of handling the wide bandwidth of the electromechanical driver. Bandwidth refers to the width of the resonance at half of its maximum power. For example, if the ultrasonic medical device is driven at a resonant frequency and the drive frequency is adjusted to obtain half of the peak power, this is referred to as half width and is how bandwidth is defined.

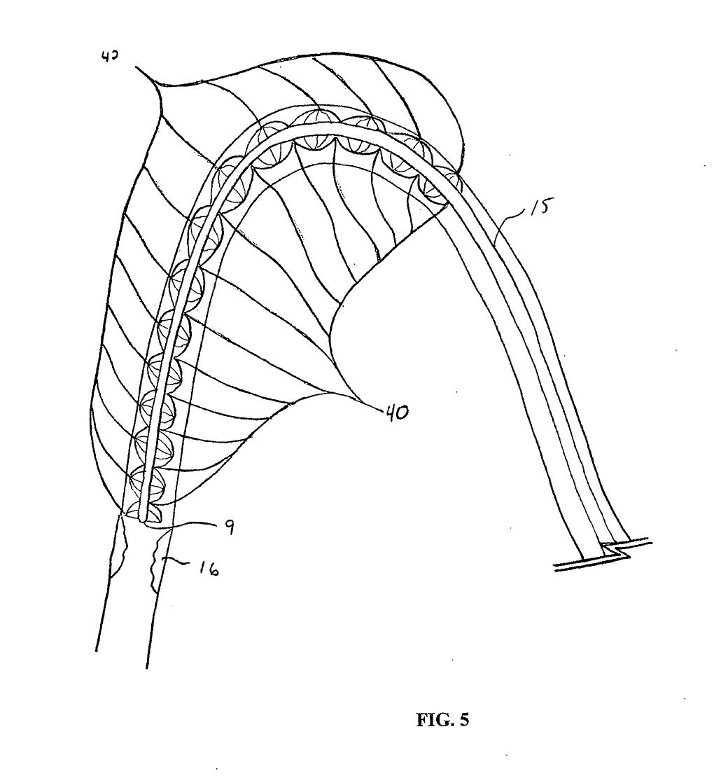

FIG. 5 shows an ultrasonic probe 15 of the present invention showing a plurality of transverse nodes 40 and a plurality of transverse anti-nodes 42 while in communication with a biological material 16 in a vasculature of a body. In FIG. 5, the ultrasonic probe 15 follows the curved path of the vasculature and ultrasonic probe 15 delivers ultrasonic energy around the bend in the vasculature. The plurality of transverse anti-nodes 42 are located along the longitudinal axis of the ultrasonic probe 15 before the bend in the vasculature, along the bend in the vasculature and after the bend in the vasculature. The variable frequency drive of the present invention varies the drive frequency to ensure that ultrasonic energy is transmit along the length of the probe including the portion after the bend to ablate the biological material 16. As discussed previously, the tortuous paths of the vasculature cause problems with a resonant ultrasonic system where the ultrasonic probe is unable to deliver sufficient ultrasonic energy to the biological material.

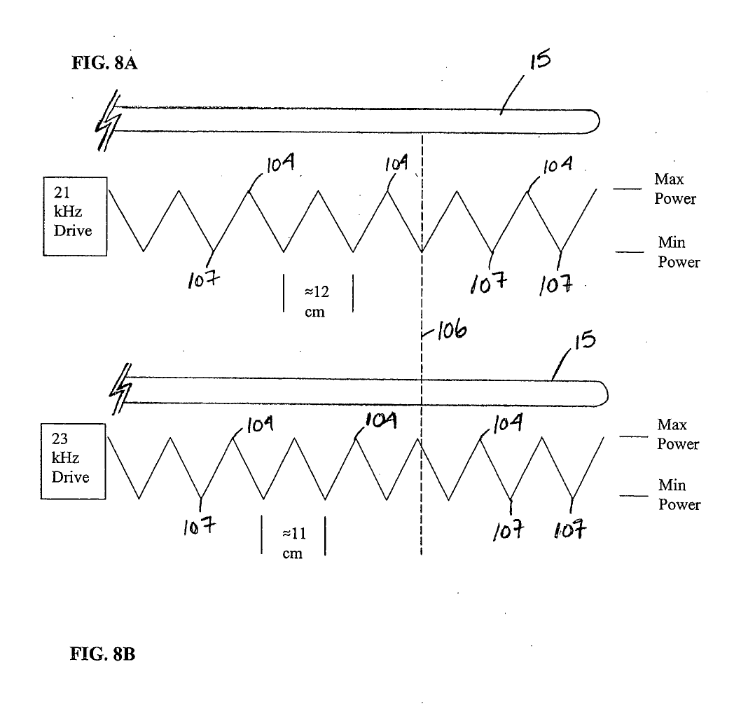

FIG. 8A and FIG. 8B show where changing the operating frequency of the ultrasonic probe 15 provides a delivery of adequate ultrasonic energy to ablate the biological material 16. In many cases, moving the ultrasonic probe 15 to a more favorable position is not possible. FIG. 8A and FIG. 8B shows the effect of bending the ultrasonic probe 15 at various locations versus energizing the ultrasonic probe 15 at two different frequencies.

FIG. 8A and FIG. 8B illustrate the power distribution along the active section of the ultrasonic probe 15 when the probe is placed in a bend in the vasculature, as the bend location is varied from the proximal end 31 to the distal end 24. The active section power varies from a peak 104, representative of a maximum power, to a trough 107, representative of a minimum power. Note that the peaks 104 and the troughs 107 of power in a bent configuration are not the same as the transverse nodes 40 and the transverse anti-nodes 42. A bend location 106 is shown to illustrate the effects of changing the operating frequency of the ultrasonic probe 15. The peaks 104 represent areas along the longitudinal axis of the ultrasonic probe 15 where the ultrasonic probe 15 may be significantly bent and still produce significant power. The troughs 107 represent areas where if the ultrasonic probe 15 is significantly bent, the power drops significantly. As shown in FIG. 8A, the bend location 106 coincides with minimum power at the trough 107 for the ultrasonic probe 15 operating at an example frequency of 21 kHz. By changing the operating frequency to a different example frequency of 23 kHz, the same bend location 106 coincides with an approximately maximum power as shown in FIG. 8B. Changing the frequency also changes the distance between adjacent troughs 107 or adjacent peaks 104. For example, in FIG. 8A, the example frequency of 21 kHz causes the distance between adjacent troughs 107 or adjacent peaks 104 to be about 12 cm. In FIG. 8B, the example frequency of 23 kHz causes the distance between adjacent troughs 107 or adjacent peaks 104 to be about 11 cm.

The ultrasonic probe 15 is placed in communication with the biological material 16 and the ultrasonic energy source 99 is activated. The horn creates a transverse wave along at least a portion of the longitudinal axis of the ultrasonic probe 15 through a nonlinear dynamic buckling of the ultrasonic probe 15. As the transverse wave is transmitted along the longitudinal axis of the ultrasonic probe 15, a transverse ultrasonic vibration is created along the longitudinal axis of the ultrasonic probe 15. The ultrasonic probe 15 is vibrated in a transverse mode of vibration. The transverse mode of vibration of the ultrasonic probe 15 differs from an axial (or longitudinal) mode of vibration disclosed in the prior art. The transverse ultrasonic vibrations along the longitudinal axis of the ultrasonic probe 15 create a plurality of transverse nodes and a plurality of transverse anti-nodes along a portion of the longitudinal axis of the ultrasonic probe 15.

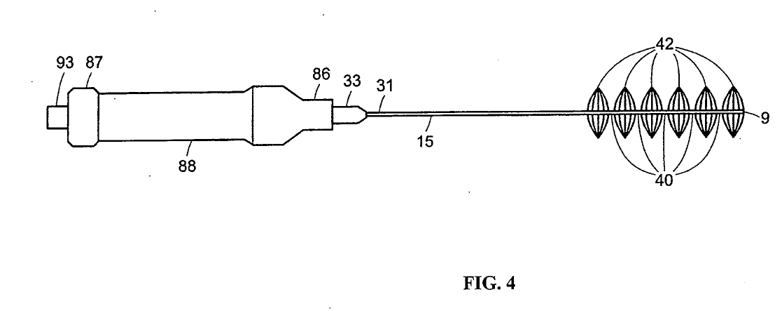

FIG. 4 shows the ultrasonic probe 15 of the present invention having a plurality of transverse nodes 40 and a plurality of transverse anti-nodes 42 along a portion of the longitudinal axis of the ultrasonic probe 15. The transverse nodes 40 are areas of minimum energy and minimum vibration. The transverse anti-nodes 42, or areas of maximum energy and maximum vibration, occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and transverse anti-nodes 42 is a function of the frequency, and can be affected by tuning the ultrasonic probe 15. In a properly tuned ultrasonic probe 15, the transverse anti-nodes 42 will be found at a position one half of the distance between the transverse nodes 40 located adjacent to each side of the transverse anti-nodes 42.

The transverse wave is transmitted along the longitudinal axis of the ultrasonic probe 15 and the interaction of the surface of the ultrasonic probe 15 with the medium surrounding the ultrasonic probe 15 creates an acoustic wave in the surrounding medium. As the transverse wave is transmitted along the longitudinal axis of the ultrasonic probe 15, the ultrasonic probe 15 vibrates transversely. The transverse motion of the ultrasonic probe 15 produces cavitation in the medium surrounding the ultrasonic probe 15 to ablate the biological material 16. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the biological material 16, while having no damaging effects on healthy tissue.

The biological material 16 is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

The transverse ultrasonic vibration of the ultrasonic probe 15 results in a portion of the longitudinal axis of the ultrasonic probe 15 vibrated in a direction not parallel to the longitudinal axis of the ultrasonic probe 15. The transverse vibration results in movement of the longitudinal axis of the ultrasonic probe 15 in a direction approximately perpendicular to the longitudinal axis of the ultrasonic probe 15. Transversely vibrating ultrasonic probes for biological material ablation are described in the Assignee's U.S. Pat. No. 6,551,337; U.S. Pat. No. 6,652,547; U.S. Pat. No. 6,660,013; and U.S. Pat. No. 6,695,781, which further describe the design parameters for such an ultrasonic probe and its use in ultrasonic devices for ablation, and the entirety of these patents are hereby incorporated herein by reference.

As a consequence of the transverse ultrasonic vibration of the ultrasonic probe 15, the biological material 16 destroying effects of the ultrasonic medical device 11 are not limited to those regions of the ultrasonic probe 15 that may come into contact with the biological material 16. Rather, as a section of the longitudinal axis of the ultrasonic probe 15 is positioned in proximity to the biological material 16, the biological material 16 is removed in all areas adjacent to the plurality of energetic transverse nodes 40 and transverse anti-nodes 42 that are produced along the portion of the length of the longitudinal axis of the ultrasonic probe 15, typically in a region having a radius of up to about 6 mm around the ultrasonic probe 15.

A novel feature of the present invention is the ability to utilize ultrasonic probes 15 of extremely small diameter compared to prior art probes, without loss of efficiency, because the biological material fragmentation process is not dependent on the area of the probe tip 9. Highly flexible ultrasonic probes 15 can therefore be designed for facile insertion into biological material areas or extremely narrow interstices that contain the biological material 16. Another advantage provided by the present invention is the ability to rapidly move the biological material 16 from large areas within cylindrical or tubular surfaces.

The variable frequency drive of the ultrasonic medical device 11 of the present invention operates to drive the ultrasonic medical device 11 one frequency at a time. As the drive frequency changes, the ablation effects of the ultrasonic probe 15 are modified. An ultrasonic probe of the ultrasonic medical device of the present invention comprises many transverse modes of vibration. For example, for an ultrasonic probe having a length of approximately one hundred thirty five centimeters and a diameter of approximately eighteen thousandths of an inch, a longitudinal resonance of the ultrasonic probe 15 occurs every approximately 1500 hertz. Approximately every 200 hertz to approximately 140 hertz, a transverse resonance of the ultrasonic probe 15 occurs. Therefore, as the drive frequency is modified, it is easier to change the frequencies to find a transverse resonance than a longitudinal resonance.

In one embodiment of the present invention, the variable frequency drive of the present invention is an open loop drive that allows for continuous variation of the frequency on the transducer without knowing what is coming back from the ultrasonic probe 15. In the open loop drive configuration, the frequency is varied in a known useful range without feedback. The operating frequency range is predetermined by manufacturing tolerances and specifications, and each transducer would operate in the same range. The ultrasonic energy source 99 can programmed for the variable frequency drive without any feedback. In this embodiment, the probe operates between frequencies where ablation of a biological material occurs while at other times, the probe operates between frequencies where ablation of a biological material does not occur. In this embodiment, the ultrasonic energy source 99 does not perform a pre-operation scan.

The functionality aspects of the open loop drive configuration of the variable frequency operation of the ultrasonic medical device 11 of the configuration are best understood by an example similar to the that shown in FIG. 8A and FIG. 8B. Assuming a 21 kilohertz (kHz) drive produces approximately 10½ cycles of interference pattern (i.e., a transverse node/transverse anti-node pattern separated by approximately 10½ centimeters) on the ultrasonic probe 15, a 23 kHz drive produces approximately 11½ cycles of interference pattern on the ultrasonic probe 15 and a 25 kHz drive produces approximately 12½ cycles of interference pattern on the ultrasonic probe 15, the particular bent configuration of the ultrasonic probe 15 affects the transverse ultrasonic vibrations and biological material ablation effects of the ultrasonic probe 15. For example, in a certain use scenario, it can be speculated that the ultrasonic probe 15 is bent in a specific manner such that the ultrasonic probe 15 does not produce transverse ultrasonic vibrations to ablate the biological material and propagate the ultrasonic energy around the bend at the 21 kHz operating frequency. However, at the 23 kHz and 25 kHz operating frequencies, the ultrasonic probe 15 does produce transverse ultrasonic vibrations to ablate the biological material and propagate the ultrasonic energy around the bend. In the open loop drive configuration of the variable frequency drive of the ultrasonic medical device 11 of the configuration, the operating frequency is slowly modulated in the range of approximately 20 kHz to approximately 26 kHz, thereby producing transverse ultrasonic vibrations and biological material ablation effects of the ultrasonic probe two-thirds of the time. Conversely, prior art resonant systems operate at only one frequency and would not produce biological material destroying effects of the ultrasonic probe.

In another embodiment of the present invention, the variable frequency drive of the ultrasonic medical device 11 of the present invention is operated in a closed loop obtaining real-time feedback from the ultrasonic probe 15 in order to modify the frequency to a frequency where ablation of a biological material 16 occurs. In this embodiment of the present invention, the loop is closed and a search is done for various parameters, including, but not limited to, the relative phase of the drive signal with respect to the feedback signal and the rate of change of this phase relationship with respect to a change in drive frequency, which help the ultrasonic energy source 99 to decide which frequency range to sweep in.

In one embodiment of the present invention, the ultrasonic medical device 11 of the present invention searches for a frequency where ablation of the biological material 16 occurs by searching the phase angles of the signal that comes back. Despite the drive being a resonant, the ultrasonic probe 15 and the rest of the ultrasonic medical device 11 does have resonances that are sensed based on feedback either from the current and voltage of the driver or from a separate microphone element in the ultrasonic medical device 11.

In another embodiment of the present invention, the ultrasonic medical device 11 of the present invention searches for a frequency where ablation of the biological material 16 occurs by detecting for cavitation based on wide band random noise which is created. In this embodiment of the present invention, an additional transducer comprising a microphone is used to pick up the reflective wave of the ultrasonic probe 15. When cavitation occurs, a random signal is produced to help identify the frequency where ablation of a biological material 16 occurs. When operation in a transverse mode of vibration occurs, there are many different frequencies that are excited at the same time. Operation in a transverse mode of vibration produces a specific noise that is picked up through the microphone.

The variable frequency drive of the ultrasonic medical device 11 of the present invention operates to vibrate the ultrasonic probe 15 in a direction transverse to the longitudinal axis. The variable frequency drive improves the ablation effects of the ultrasonic probe 15 when flexing the ultrasonic probe 15 along the bend since the variable frequency drive enables operation at a range of frequencies, thereby increasing the probability of operating the ultrasonic probe 15 in a transverse mode of operation since there are more transverse modes in a given range of frequencies than there are longitudinal modes of vibration.

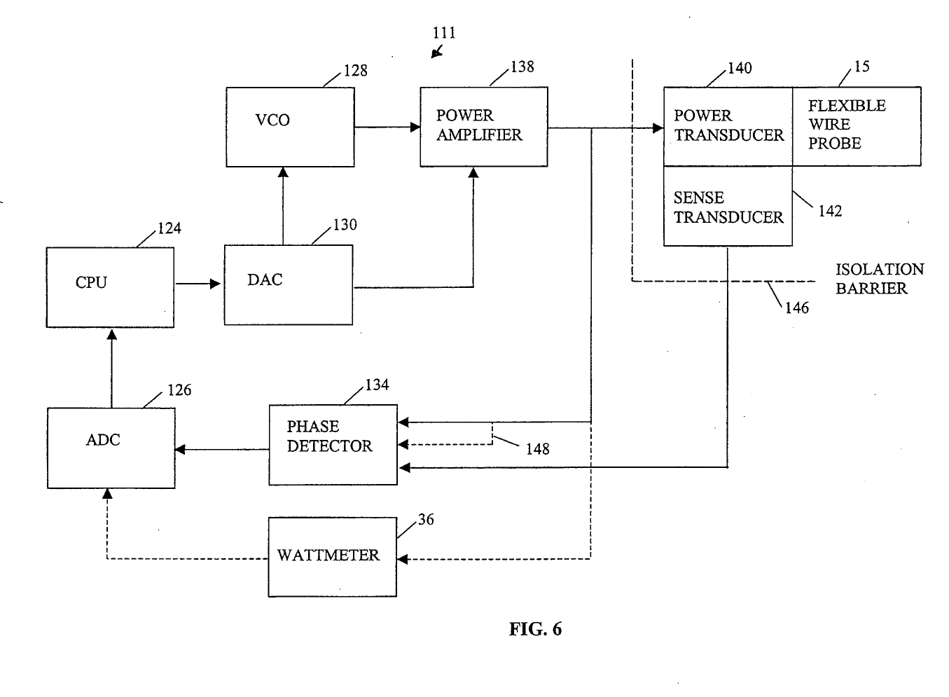

FIG. 6 is a block diagram of a preferred embodiment of the present invention where a system 111 of the ultrasonic medical device 11 uses phase analysis feedback. The system 111 is powered from an alternating current (AC) source (not shown). A central processing unit (CPU) 124 is pre-programmed to produce signals that set the frequency and amplitude of the ultrasonic drive signal based on feedback obtained from other functional blocks in the system 111. A digital to analog converter (DAC) 130 under control of the CPU 124 produces analog signals which set the output frequency of a voltage controlled oscillator (VCO) 128 and the amplitude of the drive signal produced by a power amplifier 138. The drive signal is electrically isolated via an isolation barrier 146 before being sent to the transducer assembly consisting of a power transducer 140, a sense transducer 142, and the ultrasonic probe 15 to produce ultrasonic acoustic energy. The sense transducer 142 is used to provide feedback for the system. The output signal from the sense transducer 142 must be isolated via the isolation barrier 146 before it is used by the system.

A phase detector 134 is used to compare the phase of the output voltage of the power amplifier 138 with the phase of the output voltage of the sense transducer 142 according to the following equations:

$${F}_{i n} = \vert {F}_{i n} \vert ( cos ( {ω}_{0} t + {φ}_{1} ) )$$

$${F}_{out} = \vert {F}_{out} \vert ( cos ( {ω}_{0} t + {φ}_{2} ) )$$

$$φ = {φ}_{1} - {φ}_{2} = arc cos ( \frac{{F}_{i n}}{\vert {F}_{i n} \vert} ) - arc cos ( \frac{{F}_{out}}{\vert {F}_{out} \vert} )$$

Where:

Fᵢₙ is the input function (e.g., voltage drive)

Fₒᵤₜ is the output function (e.g., sense transducer voltage)

t is the independent variable time

φ is the detected phase

ω₀ is the frequency of drive

The output of the phase detector 134 is digitized by an analog to digital converter (ADC) 126 and sent to the CPU 124. This feedback path is used to determine the frequencies at which various desirable and undesirable resonances occur in the ultrasonic probe 15 (part of a Transducer Assembly 140). The phase difference between the drive signal's voltage and the phase of the voltage signal returned from a sense transducer element may be used to locate frequencies of operation where the ultrasonic probe 15 can perform useful work. As the operating frequency is swept within the allowed frequency band, various mechanical resonances in the ultrasonic probe 15 will be excited.

Longitudinal resonances occur in the ultrasonic probe 15 according to the equation:

$$Δ f = \frac{c}{2 L}$$

Where:

Δf is the frequency spacing between longitudinal resonances

c is the longitudinal wave speed in the medium

L is the length of the ultrasonic probe

For an ultrasonic probe 15 comprising titanium with a length of 135 cm, this equates to a longitudinal resonance about every 1800 Hz.

Transverse resonances occur in the ultrasonic probe 15 according to the following equation:

$${f}_{n} = \frac{π {Kc ( 2 n - 1 )}^{2}}{8 {L}^{2}}$$

Where:

fₙ is the frequency of the nth transverse mode

K is the radius of gyration of the cross-section (which for a circular cross-section is d/4 where d is the diameter of the ultrasonic probe)

c is the longitudinal wave speed in the medium

L is the length of the ultrasonic probe

The frequency spacing around any frequency may be determined from the above formula by taking the difference between two consecutive modes. For the ultrasonic probe 15 comprising titanium with a length of 135 cm, this equates to a transverse resonance every 140 Hz at 10 kHz. As these longitudinal and transverse resonances are excited, the phase relationship between the drive signal and the returned signal (drive current or microphone element voltage) are disturbed. Longitudinal resonances cause large disturbances in the phase, and transverse resonances cause small disturbances in the phase. The following equations describe decision rules:

$$\frac{\partial φ}{\partial ω} > M , longitudinal mode$$

$$\frac{\partial φ}{\partial ω} < N , transverse mode$$

Where M is an empirically determined slowest rate of change for longitudinal mode and N is an empirically determined fastest rate of change for transverse mode.

By mapping the phase vs. frequency as the frequency is swept, the frequencies which are likely to perform useful work may be determined and excited for a given period of time before moving to a different frequency. Also, the efficacy of the ultrasonic medical device 11 at a given drive frequency may be determined by quantifying the amount of a phase jitter present in the signal returned from the sense transducer 142. Even when the ultrasonic probe 15 is excited by a single frequency, the resulting motion of the ultrasonic probe 15 causes various other frequencies and therefore phase jitter to be present in the signal returned from the sense transducer 142. During product development, phase jitter of operating probes are quantified under various conditions (for example: various efficiencies of power delivery to the target area). This information is programmed into the CPU memory. During operation, the frequency of power delivery is adjusted to various frequencies within the allowed frequency band. At each operating frequency the signal returning from the sense transducer element is analyzed and its jitter is quantified. Based on the results of the comparison, a judgement may be made with respect to the particular frequency being used. The following equations describe the decision rule:

$$\frac{\partial φ}{\partial t} > {E}_{D}$$

Where E_{D} is the empirically determined minimum phase jitter associated with efficacious power delivery at a specified drive voltage D.

If it is determined that this frequency is performing useful work, this frequency may be used to deliver useful energy to the ultrasonic probe 15 for a given period of time before moving to a different frequency. If it is determined that this frequency is not performing useful work, the system can immediately move to and test operation at a different frequency.

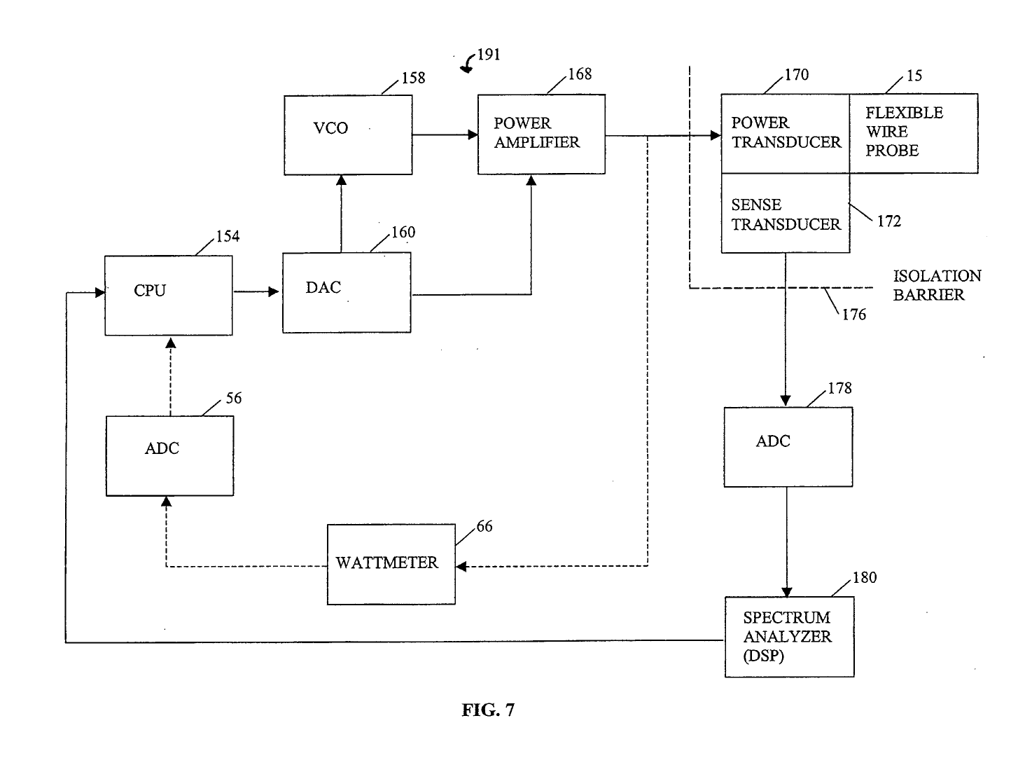

FIG. 7 is a block diagram of an alternative embodiment of the present invention where a system 191 of the ultrasonic medical device uses spectrum analysis feedback. The system is powered from an alternating current (AC) source (not shown). A central processing unit (CPU) 154 is pre-programmed to produce signals that set the frequency and amplitude of the ultrasonic drive signal based on feedback obtained from other functional blocks in the system. A digital to analog converter (DAC) 160 under control of the CPU 154 produces analog signals which set the output frequency of the voltage controlled oscillator (VCO) 158 and the amplitude of the drive signal produced by a power amplifier 168. The drive signal is electrically isolated via an isolation barrier 176 before being sent to the transducer assembly consisting of a power transducer 170, a sense transducer 172, and the ultrasonic probe 15 to produce ultrasonic acoustic energy. The sense transducer 172 is used to provide feedback for the system. The output signal from the sense transducer 172 must be isolated via an isolation barrier 176 before it is used by the system. The signal is digitized via an analog to digital converter (ADC) 178 and passed to the spectrum analyzer 180. The spectrum analyzer 180 provides information regarding the frequency spectrum of the sense transducer's output signal to the CPU 154 which allows the CPU 154 to determine the system's efficacy at the present drive signal frequency. Based on this feedback, the CPU 154 will either continue to drive the transducer assembly at the present frequency, or move to a different frequency and determine the system's efficacy at the new frequency. The sense transducer 172 in the device produces an output signal that contains information relating to the performance of the ultrasonic probe 15. Even when the ultrasonic probe 15 is excited by a single frequency, the resulting motion of the ultrasonic probe 15 causes various other frequencies to be present in the ultrasonic probe 15. During product development, spectra of operating probes are gathered under various conditions (for example: various efficiencies of power delivery to the target area). The spectra (or the important characteristics of the spectra) that are associated with optimal performance are stored in memory in the CPU 154. During operation, the frequency of power delivery is adjusted to various frequencies within the allowed frequency band. At each operating frequency the signal returning from the sense transducer element 172 is analyzed and its spectrum (or the important characteristics of the spectrum) is compared to the previously gathered probe spectra. Based on the results of the comparison, a judgement may be made with respect to the particular frequency being used. If it is determined that this frequency is performing useful work, this frequency may be used to deliver useful energy to the ultrasonic probe 15 for a given period of time before moving to a different frequency. If it is determined that this frequency is not performing useful work, the system can immediately move to and test operation at a different frequency.

A wattmeter 36, 66 may also be present in the system in order to provide feedback to the CPU 124, 154 via an analog digital converter (ADC) 126, 56. The feedback obtained from the wattmeter 36, 66 may be used to avoid spending operating time driving the system at frequencies where the transducer cannot provide energy to the ultrasonic probe 15. The feedback may also allow for adjustment of the amplitude of the drive signal in order to more closely control power delivery. The wattmeter 36, 66 operates according to the following equation:

$$Average Power , P = {\int }_{{T}_{0}}^{{T}_{0} + \frac{2 π}{{ω}_{0}}} \frac{{ω}_{0}}{2 π} V \times I d t$$

Where

T₀ is an arbitrary fixed time

Drive voltage, V=A cos(ω₀t+φ)

Drive current, I=B cos(ω₀t+φ)

This wattmeter 36, 66 would not assist with the fine adjustments of frequency, it would serve only as a gross measure of power delivered. It could not discriminate between useful power and power which does no useful work.

In an embodiment of the present invention, the system uses a phase analysis feedback source. The phase difference between the drive signal's voltage and a current 148, rather than the phase between the drive signal's voltage and the phase of the voltage signal returned from a sense transducer element, may be used to locate frequencies of operation where the flexible probe can perform useful work.

The closed loop operation may be Scan Closed Loop/Run Open Loop or Run Closed Loop. These two types of closed loop operation are similar. In an embodiment of the present invention, the closed loop mode of operation is Scan Closed Loop/Run Open Loop where there are two distinct operating conditions: scanning and delivering energy. In another embodiment of the present invention, the closed loop mode of operation is Run Closed Loop where useful energy is being delivered to the flexible probe simultaneously with the frequency analysis. Those skilled in the art will recognize that other closed loop operations known in the art are within the spirit and scope of the invention.

In an embodiment of the present invention, the open loop mode of operation has a drive frequency that is slowly varied (modulated) within the allowed frequency band. The frequency modulation is a prescribed function of time (e.g., sinusoidal), and the modulation signal band is limited to less than about 100 Hz.

In an embodiment of the present invention, there is simultaneous excitation at multiple frequencies: Several VCOs may be used to simultaneously drive the power transducer at several frequencies in order to maximize delivery of energy to the target area.

In an alternative embodiment of the present invention, the ultrasonic probe 15 is vibrated in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985, and the entirety of this application is hereby incorporated herein by reference.

In another embodiment of the present invention, the ultrasonic probe 15 is vibrated in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active section of the ultrasonic probe 15, creating a plurality of nodes and a plurality of anti-nodes along the active section that result in cavitation in a medium surrounding the ultrasonic probe 15. The active section of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active section of the ultrasonic probe 15 is vibrated in a direction not parallel to the longitudinal axis of the ultrasonic probe 15 while equally spaced points along the longitudinal axis of the ultrasonic probe 15 vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898, and the entirety of this application is hereby incorporated herein by reference.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An ultrasonic medical device comprising:
an elongate wire having a proximal end, a distal end and a longitudinal axis therebetween;
a proximal end portion of the elongate wire being engaged to a transducer, the elongate wire being configured such that a transverse ultrasonic vibration is created along at least a portion of the longitudinal axis of the elongate wire when ultrasonic energy is transmitted to the elongate wire by the transducer; and
a control unit adapted to sequentially drive the elongate wire at multiple different frequencies during use, wherein driving the elongate wire at the multiple different frequencies during use allows for the ultrasonic energy to propagate around a bend of the elongate wire, the control unit being further adapted to determine at which of the multiple different frequencies a transverse resonance occurs in the elongate wire.

2. The ultrasonic medical device of claim 1 wherein the elongate wire comprises a material that allows the elongate wire to be bent, deflected and flexed.

3. The ultrasonic medical device of claim 1 wherein the transducer obtains a broadband signal to drive the elongate wire and produce power over a broad range of frequencies.

4. The ultrasonic medical device of claim 1 wherein the transducer operates at frequencies away from the resonant frequencies of the elongate wire.

5. The ultrasonic medical device of claim 1 wherein a plurality of transverse resonances of the elongate wire are excited.

6. The ultrasonic medical device of claim 1 wherein a longitudinal resonance of the elongate wire is avoided.

7. The ultrasonic medical device of claim 1 wherein the transducer allows for uniform power output over the multiple different frequencies.

8. The ultrasonic medical device of claim 1 wherein the transducer is a magnetostrictive mechanism.

9. The ultrasonic medical device of claim 1 wherein the transducer is a voicecoil mechanism.

10. The ultrasonic medical device of claim 1 wherein the transducer is a pneumatic mechanism.

11. The ultrasonic medical device of claim 1 further comprising a broadband ultrasonic energy source connected to the transducer.

12. The ultrasonic medical device of claim 1 wherein the transverse ultrasonic vibration generates a plurality of transverse nodes and a plurality of transverse anti-nodes along at least a portion of the longitudinal axis of the elongate wire.

13. The ultrasonic medical device of claim 1 wherein the elongate wire is driven in an open loop configuration over the multiple different frequencies.

14. The ultrasonic medical device of claim 1 wherein the elongate wire is driven in a closed loop configuration over the multiple different frequencies.

15. The ultrasonic medical device of claim 1 wherein the transducer drives the elongate wire over the multiple different frequencies causing a longitudinal ultrasonic vibration along at least a portion of the longitudinal axis of the elongate wire.

16. The ultrasonic medical device of claim 1 wherein the transducer drives the elongate wire over the multiple different frequencies causing a torsional ultrasonic vibration along at least a portion of the longitudinal axis of the elongate wire.

17. The ultrasonic medical device of claim 1 wherein the elongate wire is disposable.

18. The ultrasonic medical device of claim 1 wherein the elongate wire contains a super-elastic alloy.

19. An ultrasonic medical device comprising:
an elongate wire having a proximal end, a distal end and a longitudinal axis between the proximal end and the distal end;
a transducer adapted to convert electrical energy into mechanical energy;
a proximal end portion of the elongate wire being engaged to the transducer, the elongate wire being configured such that a transverse ultrasonic vibration is created along at least a portion of the longitudinal axis of the elongate wire when the mechanical energy is transmitted to the elongate wire by the transducer, and
a control unit adapted to sequentially drive the ultrasonic at multiple different frequencies with an approximately uniform power output during use, the control unit being further adapted to determine at which of the multiple different frequencies a transverse resonance occurs in the elongate wire.

20. A method comprising:
flexing an elongate wire along a bend of a vasculature of a body, the elongate wire having a proximal end, a distal end and a longitudinal axis therebetween;
activating an ultrasonic energy source engaged to a proximal end portion of the elongate wire to generate a transverse ultrasonic vibration along at least a portion of the longitudinal axis of the elongate wire;
sequentially driving the elongate wire at multiple different frequencies to allow the transverse ultrasonic vibration to propagate along a bent region of the elongate wire disposed in the bend of the vasculature; and
determining at which of the multiple different frequencies a transverse resonance occurs in the elongate wire.
